(19) United States
(12) Patent Application Publication
(10) Pub. No.: US 2010/0104652 A1
(43) Pub. Date: Apr. 29, 2010

(54) Use of advanced nanomaterials for increasing sepecific cell functions

(75) Inventors: Alexandru S. Biris, Little Rock, AR (US); Meena Mahmood, Little Rock, AR (US); Peder Jensen, Little Rock, AR (US)

Correspondence Address: FOLEY AND LARDNER LLP;SUITE 500, 3000 K STREET NW, WASHINGTON, DC, 20007, US

(73) Assignee: UNIVERSITY OF ARKANSAS

(21) Appl. No.: 12/588,679

(22) Filed: Oct. 23, 2009

Related U.S. Application Data
(60) Provisional application No. 61/197,424, filed on Oct. 27, 2008.

(51) Int. Cl. A61K 9/50; C12N 5/02; C12N 5/00; C04B 35/00; C01B 13/14; D01F 9/12; C01B 15/16; C01G 25/02; C22C 5/06; C22C 5/02

(52) U.S. Cl. 424/490; 435/375; 435/402; 501/1; 423/592.1; 423/447.1; 423/305; 423/608; 420/501; 420/507; 977/773; 977/742; 977/906

(57) ABSTRACT

Disclosed herein are methodologies and compositions for enhancing cellular functions, which can be used in a variety of biological applications.

### CROSS-REFERENCE TO RELATED APPLICATION

This application claims the benefit of U.S. Provisional Application No. 61/197,424, filed Oct. 27, 2008.

### INTRODUCTION

Bone is a living, growing tissue comprised predominantly of collagen, hydroxyapatite crystals, and cells and organic material. Collagen provides a soft framework, whereas calcium hydroxyapatite strengthens and hardens the framework. This combination of collagen and calcium hydroxyapatite makes bone strong yet flexible enough to withstand stress.

Bone formation occurs by the concurrent activity of osteoblasts and osteoclasts, as well as the addition of minerals and salts/crystals. Osteoblasts deposit new bone while osteoclasts absorb old bone, such that total bone mass remains constant at any given time under most conditions.

Bone regeneration and the generation of new tissue is important for treating various diseases and reconstructive surgery. For example, bone grafts can be used to promote healing of fractures, and to augment bone during plastic and reconstructive surgery. Consequently, bone allograft and bone substitutes play an important role in bone grafting, bone repair, bone replacement, and/or bone-implant fixation purposes.

### SUMMARY

Embodiments herein include but are not limited to methods, devices, compositions, kits, materials, tools, instruments, reagents, products, compounds, pharmaceuticals, arrays, computer-implemented algorithms, and computer-implemented methods.

In one aspect, there is provided a method for regulating a cellular function, comprising (a) combining a material and a nanoparticle composition to form a material system, wherein said combining is by coating or binding; and (b) delivering said material system to a cell type or tissue. In one embodiment, the material is for a biomedical application. In another embodiment, the material is at a nano, micro, or macro scale. In another embodiment, the nanoparticle composition comprises gold, metal, metal oxide, polymer, titanium dioxide, silver, carbon nanotubes, hydroxyapatite, quantum dots, crystals, salts, ceramic materials, magnetic materials, or any combination thereof. In some embodiments, the tissue is hard tissue or soft tissue.

In another embodiment, the nanoparticle composition comprises a plurality of materials. In a further embodiment, the plurality comprises (a) a core made of one material; and (b) at least one layer surrounding said core, wherein said layer comprises a material that is not the same as said core.

In another embodiment, the nanoparticle composition comprises material having a size from about 0.5 nm to about 50 mm. In a further embodiment, the material has a size from about 50 microns to 50 mm.

In another embodiment, the material is coated with at least one layer of coating that delivers a drug or bioactive agent, wherein said drug or bioactive agent prevents oxidation, prevents or treats infection, increases bio-compatibility, increases or decreases a cell function, promotes cell adhesion and proliferation, reduces toxicity, or promotes binding with a biological or non-biological system.

In another embodiment, said delivering is by injection, epidermal translation, inhalation, surgical delivery, topical application, oral administration or any other method that targets a desired cell type or tissue.

In another embodiment, said cellular function is at least one of bone formation, protein synthesis, gene expression, cell proliferation, mitosis, DNA transcription, hormone production, enzyme production, cell death, gene delivery, or drug delivery.

In another embodiment, said material is a natural or synthetic polymer, metal, metal oxide, metal nitride, borate, ceramic, allograft hard tissue, allograft soft tissue, xenograft hard tissue, xenograft soft tissue, carbon nanostructure, glasses, or natural, or biocompatible material. In a further embodiment, said carbon nanostructure is a single walled carbon nanotube, double walled carbon nanotube, multi walled carbon nanotube, nanofiber, fullerene, or grapheme.

In another embodiment, said material is coated with an oxide, nitride, borate, polymer, ceramic, zirconia, metal, metal oxide, carbon, or graphitic material.

In another embodiment, said material system is capable of preventing or treating an infection. In a further embodiment, said infection is a bacterial, fungal, prion, parasite, or viral infection.

In another aspect, there is provided a method for regulating a cellular function, comprising delivering nanoparticles in a solution, aerosol, gel, cream, paste to a cell type or tissue. In one embodiment, said delivery is by injection, inhalation, oral administration, or topical application.

In another aspect, there is provided a device for regulating at least one cellular function, wherein said device comprises a material system. In one embodiment, said device is an implant, graft, needle, catheter, dental implant, gel, cream, injectible, orthopedic implant, prosthetic, cardiovascular stent, defibrillator/pacemaker, medical tube, tissue engineering matrix or scaffold. In a further embodiment, said scaffold is a tissue-forming scaffold.

In another aspect, there is provided a device comprising nanoparticles, wherein said nanoparticles are layered throughout and/or on the surface of said device. In one embodiment, said device is biodegradable and/or biocompatible. In another embodiment, said nanoparticles are released from said device as each layer degrades.

In another aspect, there is provide a composition for promoting tissue growth, wherein said composition comprises nanoparticles. In one embodiment, said nanoparticles are layered in a surface coating covering. In another embodiment, said nanoparticles comprise gold, silver, metals, metal oxides, oxides, polymers, ceramics, carbon nanotubes, or hydroxyapatite.

In one aspect, there is provided a coating for an implant; wherein said coating comprises nanoparticles. In one embodiment, said implant is polymeric, metal, metal alloy, shape memory metal, metal oxide, oxide, ceramic, zirconia, or hydroxyapatite.

In another aspect, there is provided a method for regulating a cellular function, comprising (a) linking at least one nanoparticle to an agent to create a stimulating agent; and (b) delivering said stimulating agent to a cell type or tissue. In one embodiment, said agent comprises a protein, growth factor, antibody, amino acids, polymers, drug(s), hormone, nucleic acid, peptide, or enzyme. In another embodiment, said cellular function is bone growth and said stimulating agent is an osteoblast stimulating agent. In one embodiment, said linking is covalent, polar covalent, ionic, sulfide, hydrogen bond, or any other linkage suitable for in vivo. In another embodiment, said growth factor is Bone Morphogenic Proteins (BMPs), Brain-Derived Neutrophic Factor (BDNF), Ciliary Neutrophic Factor (CNTF), Epidermal Growth Factor (EGF), Erythropoietin (EPO), Fibroblast Growth Factor (FGF), Granulocyte-Colony Stimulating Factor (G-CSF), Granulocyte-Macrophage Colony Stimulating Factor (GM-CSF), Growth Differentiation Factor-9 (GDF9), Hepatocyte Growth Factor (HGF), Insulin-like Growth Factor (IGF), Interleukin (IL), Leukemia Inhibitory Factor (LIF), Myostatin (GDF-8), Nerve Growth Factor (NGF), Neutrophic Factors (NT), Platelet-derived Growth Factor (PDGF), Thrombopoietin (TPO), Transforming Growth Factor alpha(TGF-α), Transforming Growth Factor beta (TGF-β), or Vascular Endothelial Growth Factor (VEGF). In another embodiment, said delivering is by injection, surgical placement, oral administration, inhalation, or transdermal application. In another embodiment, the method further comprises(c) exposing said cell or tissue comprising said stimulating agent to radiation for faster penetration. In a further embodiment, the radiation is laser radiation or electromagnetic radiation. In another further embodiment, the stimulating agent is placed in a solution, gel, paste, suspension, or aerosol, for delivery to said cell type or tissue.

### BRIEF DESCRIPTION OF THE DRAWINGS

FIG. 1 presents morphological features of osteoblast cells incubated for 24 hrs with three different nanomaterials: (a) silver nanoparticles, (b) carbon nanotubes, and (c) nanohydroxyapatite (nano-HAP). Original magnification 40×, bar=50 μm.

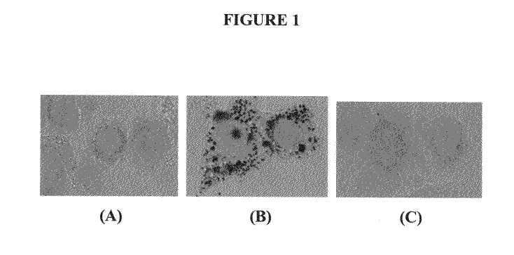

FIG. 2 illustrates the effects of nanomaterials on the concentration of Alizarin Red stain. Osteoblast cells were incubated in the presence and absence of (10 μg/ml) of silver nanoparticles (Ag-NPs), nanohydroxyapatite (nano-HAP), titanium dioxide (TiO₂) nanoparticles, and carbon nanotubes (CNTs). The experiments were completed on day 24. The results are derived from three experiments, with 6 cultures for each variable in each experiment (n=6). Alizarin Red concentration was determined by comparing the samples OD₄₀₅ with standard sample of 2 mM of ARS diluted with 1× ARS dilution buffer.

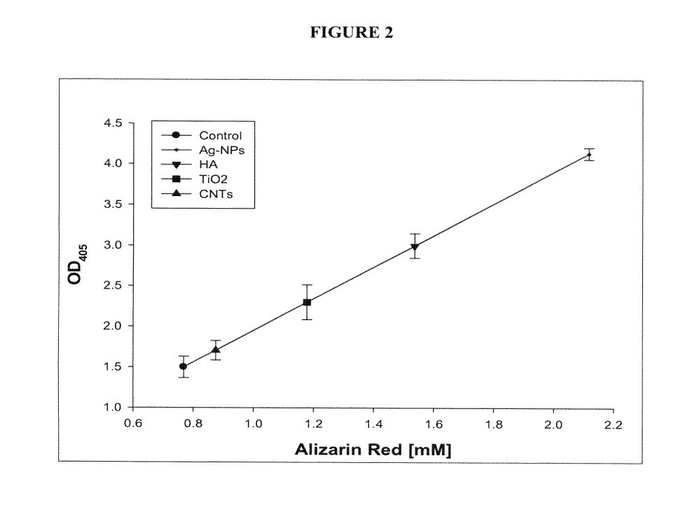

FIG. 3 illustrates the effects of silver nanoparticles on the concentration of Alizarin Red stain as a function of time. 10⁵ cells were plated per well with and without silver nanoparticles (10 μg/ml) and incubated for 6, 15, and 24 days, the results are derived from 3 experiments, with 6 cultures for each variable in each experiment. Bars represent the OD₄₀₅ which is correlated with the Alizarin Red concentration in each well.

FIG. 4 illustrates mineralized nodules formation of osteoblasts in the presence of nanomaterials stained by alizarin red S. (A) cells without nanomaterials as a control; (B) cells with Ag-NPs; (C) cells with HAP nanoparticles, (D) cells with TiO₂ nanoparticles; (E) cells with SW-CNTs. Original magnification 40×, bar=50 μm. The more red the image is, the more mineral has formed after exposing the cells to the various nanomaterials.

### DETAILED DESCRIPTION

Methodologies, materials, and devices provided herein regulate cellular functions, which can be used in a variety of biological applications. More specifically, and as described in greater detail below, the present inventors discovered that nanoparticle compositions can accelerate cell functions, such as enhanced bone growth by osteoblasts.

All technical terms used herein are terms commonly used in cell biology, biochemistry, molecular biology, and nanotechnology and can be understood by one of ordinary skill in the art to which this invention belongs. These technical terms can be found in the current editions of Molecular Cloning: A Laboratory Manual, (Sambrook et al., Cold Spring Harbor); Gene Transfer Vectors for Mammalian Cells (Miller & Calos eds.); and Current Protocols in Molecular Biology (F. M. Ausubel et al. eds., Wiley & Sons). Cell biology, protein chemistry, and antibody techniques can be found in Current Protocols in Protein Science (J. E. Colligan et al. eds., Wiley & Sons); Current Protocols in Cell Biology (J. S. Bonifacino et al., Wiley & Sons) and Current Protocols in Immunology (J. E. Colligan et al. eds., Wiley & Sons.). Reagents, cloning vectors, and kits are available from commercial vendors such as BioRad, Stratagene, Invitrogen, ClonTech, and Sigma-Aldrich Co.

Cell culture methods are described generally in the current edition of Culture of Animal Cells: A Manual of Basic Technique (R. I. Freshney ed., Wiley & Sons); General Techniques of Cell Culture (M. A. Harrison & I. F. Rae, Cambridge Univ. Press), and Embryonic Stem Cells: Methods and Protocols (K. Turksen ed., Humana Press). Other texts include Creating a High Performance Culture (Aroselli, Hu. Res. Dev. Pr. 1996) and Limits to Growth (D. H. Meadows et al., Universe Publ. 1974). Tissue culture supplies and reagents are available from commercial vendors such as Gibco/BRL, Nalgene-Nunc International, Sigma Chemical Co., and ICN Biomedicals.

Although this specification provides guidance to one skilled in the art to practice the invention including reference to technical literature, mere reference does not constitute an admission that the technical literature is prior art.

### A. Construction of Material System

The present inventors have developed material systems at nano and micro levels for regulating at least one cellular function. Cellular function includes any process a cell undergoes or participates in, including but not limited to bone formation, protein synthesis, cell repair, cell division, cell proliferation/mitosis, cell differentiation, cell death, gene expression, cell respiration, DNA transcription and drug delivery. As used herein, regulating means altering at least one of quantity, speed, rate, efficiency, quality, or target delivery. Thus, for example and in no way limiting, a material system may increase bone formation, alter apoptosis, or improve drug delivery. Regulating may also mean increase or decrease of a specific cellular function.

A material delivery system may comprise, or form from, any material suitable for delivery into a living organism. For example, and in no way limiting, a material system may be polymeric, metal, metal oxide, ceramic, carbon-composite, stainless steel, cobalt-chromium, titanium alloy, shape memory metal, tantalum, and combinations thereof. A structured surface can be defined by, or composed of, or formed of a material that includes a plurality of particles that are sintered together to form a continuous porous phase. Alternatively, a structured surface can be prepared by at least one of flame spraying, acid etching, grit blasting, casting-in, forging-in, laser texturing, micromachining, plasma treatment, ion bombardment, physical vapor deposition, or chemical vapor deposition

A material may have any shape suitable for an intended purpose, as the circumstances present. One of ordinary skill in the art will understand the need for a particular material and will appreciate the shape or morphology necessary to accomplish a particular need. Material shapes include but are not limited to spheres (filled and unfilled), squares, cylinders, cubes, pods, cones, pyramids, and filaments. The structures can be at the nano, micro, or macro level and can have a plurality of shapes and dimensions. It is envisioned that the shapes could be spherical, tubular, cylindrical, triangular, plates, hexagonal, fibrous, or any morphological shape that can interact with cells in the desired manner. Also it is possible to have a combination of structures with various shapes and structures in such a way that together or individually the system plays the desired role.

A material should be biocompatible or non-toxic with a living organism receiving said material. Biocompatibility can be accomplished by constructing a material system from material that will not interfere with a host organism's basic functions and/or coating a material's surface. In the case of a coating, a biocompatible coating can include one or more of titanium, tantalum, carbon, calcium phosphate, zirconium, niobium, hafnium, metals, metal oxides, oxides, hydroxyapatite, nano-hydroxyapatite, polymer, tissue in-growth and/or on-growth facilitating proteins, and combinations thereof. For example, if carbon is used, it may be diamond-like carbon, pyrolytic carbon, amorphous diamond-like carbon, and combinations thereof One of ordinary skill in the art would understand that a biocompatible material system is made from materials and/or coatings that are not rejected by the recipient organism and would understand how to produce or obtain such materials.

Importantly, a material system for regulating at least one cellular function comprises nanoparticles. Nanoparticle refers to a small object that behaves as a whole unit in terms of its transport and properties. Generally, nanoparticles have a size of about 0.5 nm to about 100 nm. A nanoparticle may comprise or be made from any suitable material including but not limited to metal nanoparticles (gold, silver, etc), metal oxides (titanium dioxide, ZnO, etc), polymers, carbon nanostrucutres (single, double, multi walled carbon nanotubes, graphene, fullerenes, nanofibers), hydroxyapatite, nano crystals (quantum dots, crystals, etc), salts, ceramic materials, and any combinations thereof.

It can be advantageous for a material system to be biodegradable or partially biodegradable. The system can also be released from the body or can be retained in various tissues or organs. Similarly, and in some embodiments, a material system may biodegrade in a layered approach, such that nanoparticles and other bioactive or therapeutic agents/drugs are released layer-by-layer as the material system degrades.

### B. Nanolinkage Composition

Separately or in conjunction with the inventive material systems, the present inventors contemplate regulating a cellular function with a nanolinkage composition. A nanolinkage composition comprises at least one nanoparticle linked to at least one agent. Exemplary agents include but are not limited to proteins, growth factors, hormones, antibodies, amino acids, carbohydrates, polymers, drugs, nucleic acids, and/or enzymes. The agents can be linked to a nanoparticle by any suitable means, including but not limited to covalent bond, polar covalent bond, hydrogen bond, sulfide bond, or ionic bond. The systems can be delivered by injection, epidermal translation, inhalation, direct surgical placement, or any other suitable method known in the art.

A stimulating agent comprises at least one nanoparticle linked to at least one agent. A stimulating agent may comprise a nanoparticle linked to at least one protein, growth factor, antibody, amino acid, polymer, drug, nucleic acid, hormone, and/or enzyme. For example, a stimulating agent comprising a nanoparticle linked to a bone morphogenic protein (BMP) may stimulate mesenchymal/stem cells to differentiate into an osteoblast cell and is herein referred to as an osteoblast stimulating agent.

As used herein, a growth factor is a naturally occurring substance capable of stimulating cellular growth, proliferation, and/or cellular differentiation. Generally, a growth factor is a protein or a steroid hormone, and typically acts as a signaling messenger between cells. Relevant to an enhanced cellular function, a growth factor may promote cell differentiation, cell growth, protein synthesis, and/or gene expression, each of which varies based on the particular growth factor employed. For example, bone morphogenic proteins (BMPs) stimulate bone cell differentiation, while fibroblast growth factors and vascular endothelial growth factors (VEGF) stimulate blood vessel differentiation (angiogenesis).

Non-limiting exemplary growth factors include Bone Morphogenic Proteins (BMPs), Brain-Derived Neutrophic Factor (BDNF), Ciliary Neutrophic Factor (CNTF), Epidermal Growth Factor (EGF), Erythropoietin (EPO), Fibroblast Growth Factor (FGF), Granulocyte-Colony Stimulating Factor (G-CSF), Granulocyte-Macrophage Colony Stimulating Factor (GM-CSF), Growth Differentiation Factor-9 (GDF9), Hepatocyte Growth Factor (HGF), Insulin-like Growth Factor (IGF), Interleukin (IL), Leukemia Inhibitory Factor (LIF), Myostatin (GDF-8), Nerve Growth Factor (NGF), Neutrophic Factors (NT), Platelet-derived Growth Factor (PDGF), Thrombopoietin (TPO), Transforming Growth Factor alpha(TGF-α), Transforming Growth Factor beta (TGF-β), and Vascular Endothelial Growth Factor (VEGF).

Sequences such as proteins, amino acids, nucleic acids may be naturally isolated or synthetically produced. For example, synthetic sequences, such as synthetic polypeptides may be generated using techniques well known to those of ordinary skill in the art. Recombinant DNA techniques with bacteria may be used. For instance, such polypeptides may be synthesized using any of the commercially available solid-phase techniques, such as the Merrifield solid-phase synthesis method, wherein amino acids are sequentially added to a growing amino acid chain. (Merrifield, J. Am. Chem. Soc. 85: 2149-2154, 1963). Equipment for automated synthesis of polypeptides is commercially available from suppliers such as Perkin Elmer/Applied Biosystems, Inc. (Foster City, Calif.), and may be operated according to the manufacturer's instructions. Likewise, variants of a native polypeptide may be prepared using standard mutagenesis techniques, such as oligonucleotide-directed site-specific mutagenesis (Kunkel, Proc. Natl. Acad. Sci. USA 82: 488-492, 1985). Sections of DNA sequences may also be removed using standard techniques to permit preparation of truncated polypeptides.

As used herein, the term “antibody” refers to any immunoglobulin, whether natural or wholly or partially synthetically produced. All derivatives thereof which maintain specific binding ability are also included in the term. The term also covers any protein having a binding domain which is homologous or largely homologous to an immunoglobulin binding domain. Such proteins may be derived from natural sources, or partly or wholly synthetically produced. An antibody may be monoclonal or polyclonal. An antibody may be a member of any immunoglobulin class, including any of the human classes: IgG, IgM, IgA, IgD, and IgE. As used herein, the terms “antibody fragment” or “characteristic portion of an antibody” are used interchangeably and refer to any derivative of an antibody which is less than full-length. In general, an antibody fragment retains at least a significant portion of the full-length antibody's specific binding ability. Examples of antibody fragments include but are not limited to Fab, Fab′, F(ab′)2, scFv, Fv, dsFv diabody, and Fd fragments. An antibody fragment may be produced by any means. For example, an antibody fragment may be enzymatically or chemically produced by fragmentation of an intact antibody and/or it may be recombinantly produced from a gene encoding the partial antibody sequence. Alternatively or additionally, an antibody fragment may be wholly or partially synthetically produced. An antibody fragment may optionally comprise a single chain antibody fragment. Alternatively or additionally, an antibody fragment may comprise multiple chains which are linked together, for example, by disulfide linkages. An antibody fragment may optionally comprise a multimolecular complex. A functional antibody fragment typically comprises at least about 50 amino acids and more typically comprises at least about 200 amino acids.

Any drug or therapeutic agent may be used in a nanolinkage composition. Furthermore, the drug or therapeutic agent with or without a nanolinkage composition can be placed either on the surface or within the surface coating layers via any deposition method. In some embodiments, the agent is a clinically-used drug including but not limited to an antibiotic, antifungal agent, anti-viral agent, anesthetic, anticoagulant, anti-cancer agent, inhibitor of an enzyme, steroidal agent, anti-inflammatory agent, anti-neoplastic agent, antigen, vaccine, antibody, decongestant, antihypertensive, sedative, progestational agent, anti-cholinergic, analgesic, anti-depressant, anti-psychotic, β-adrenergic blocking agent, diuretic, cardiovascular active agent, vasoactive agent, and non-steroidal anti-inflammatory agent. A drug or therapeutic agent may be a mixture of pharmaceutically active agents. For example, a local anesthetic may be delivered in combination with an anti-inflammatory agent such as a steroid. Local anesthetics may also be administered with vasoactive agents such as epinephrine. To give but another example, an antibiotic may be combined with an inhibitor of the enzyme commonly produced by bacteria to inactivate the antibiotic (e.g. penicillin and clavulanic acid.

Thus, and solely as an example, a nanolinkage composition may comprise a drug or therapeutic agent for preventing bone degradation leading to osteoporosis. In one embodiment, any class of drugs preventing osteoporosis, such as bisphosphonates, may be used in a nanolinkage composition.

### C. Delivery of Material System

An inventive material system can be delivered by any suitable method known in the art. For example, and in no way limiting, a material system can be delivered by direct injection, epidermal translation, inhalation, or direct surgical placement. Delivery can be directed to any cell type or tissue.

In one embodiment, a material system may be delivered to any eukaryotic cell or tissue of interest. In certain embodiments, a cell is a mammalian cell. Cells may be of human or non-human origin. For example, they may be of mouse, rat, or non-human primate origin. Exemplary cell types include but are not limited to endothelial cells, epithelial cells, mesenchymal cells, stem cells, muscle cells, neurons, hepatocytes, myocytes, chondrocytes, osteoblasts, osteoclasts, lymphocytes, macrophages, neutrophils, fibroblasts, keratinocytes, etc. Cells can be primary cells, immortalized cells, transformed cells, terminally differentiated cells, stem cells (e.g. adult or embryonic stem cells, hematopoietic stem cells), somatic cells, germ cells, etc. Cells can be wild type or mutant cells, e.g., they may have a mutation in one or more genes. Cells may be quiescent or actively proliferating. Cells may be in any stage of the cell cycle. In some embodiments, cells may be in the context of a tissue. In some embodiments, cells may be in the context of an organism.

Cells can be normal cells or diseased cells. In certain embodiments, cells are cancer cells, e.g. they originate from a tumor or have been transformed in cell culture (e.g. by transfection with an oncogene). In certain embodiments, cells are infected with a virus or other infectious agent. A virus may be, e.g. a DNA virus, RNA virus, retrovirus, etc. For example, cells can be infected with a human pathogen such as a hepatitis virus, a respiratory virus, human immunodeficiency virus, etc.

Cells can be cells of a cell line. Exemplary cell lines include HeLa, CHO, COS, BHK, NIH-3T3, HUVEC, etc. For an extensive list of cell lines, one of ordinary skill in the art may refer to the American Type Culture Collection catalog (ATCC®, Manassas, Va.).

In some embodiments, speed or delivery rate to a cell type and/or tissue may be increased by exposing said cell and/or tissue comprising a material system to radiation, which permits faster penetration of the host cell and/or tissue. Any suitable radiation technique may be used, including laser radiation and electromagnetic radiation.

### D. Assaying Cell Function

As disclosed herein, a material system may be used to regulate cellular functions, which can be used in a variety of biological applications. Cellular functions include but are not limited to bone formation, protein synthesis, gene expression, cell proliferation, mitosis, DNA transcription, hormone production, enzyme production, cell death, gene delivery, or drug delivery.

Depending on the particular cellular function, and as circumstances vary, one of ordinary skill in the art would know how to assay cell function using methods known in the art. For example, in the case of gene expression and detecting a level of polynucleotide expression, any method for observing polynucleotide expression can be used without limitation. Such methods include but are not limited to traditional nucleic acid hybridization techniques, polymerase chain reaction (PCR) based methods, and protein determination. Absolute measurements of the expression levels need not be made, although they can be made. Thus, the present disclosure contemplates methods for comparing differences in expression levels between samples. Comparison of expression levels can be done visually or manually, or can be automated and done by a machine, using for example optical detection means. Subrahmanyam et al., Blood. 97: 2457 (2001); Prashar et al., Methods Enzymol. 303: 258 (1999). Hardware and software for analyzing differential expression of genes are available, and can be adapted for a particular gene. See, e.g., GenStat Software and GeneExpress.™. GX Explorer.™. Training Manual, supra; Baxevanis & Francis-Ouellette, supra. Likewise, nucleic acid hybridization techniques can be used to observe polynucleotide expression. Exemplary hybridization techniques include northern blotting, Southern blotting, solution hybridization, and S1 nuclease protection assays.

Similarly, cellular function can be assayed based on protein expression levels. Proteins can be observed by any means known in the art, including immunological methods, enzyme assays and protein array/proteomics techniques. Measurement of the translational state can be performed according to several protein methods. For example, whole genome monitoring of protein—the “proteome”—can be carried out by constructing a microarray in which binding sites comprise immobilized, preferably monoclonal, antibodies specific to a plurality of proteins. See Wildt et al., Nature Biotechnol. 18: 989 (2000). Methods for making polyclonal and monoclonal antibodies are well known, as described, for instance, in Harlow & Lane, ANTIBODIES: A LABORATORY MANUAL (Cold Spring Harbor Laboratory Press, 1988).

Alternatively, proteins can be separated by two-dimensional gel electrophoresis systems. Two-dimensional gel electrophoresis is well-known in the art and typically involves isoelectric focusing along a first dimension followed by SDS-PAGE electrophoresis along a second dimension. See, e.g., Hames et al, GEL ELECTROPHORESIS OF PROTEINS: A PRACTICAL APPROACH (IRL Press, 1990). The resulting electropherograms can be analyzed by numerous techniques, including mass spectrometric techniques, western blotting and immunoblot analysis using polyclonal and monoclonal antibodies, and internal and N-terminal micro-sequencing.

Likewise, cellular function can be assayed based by staining for cellular or morphological markers associated with a particular cellular function. For example and in no way limiting, a cellular function like bone formation can be assayed by staining for calcium mineralization using Alizarin red. The more calcium, the more mineralization of collagen to produce bone via osteoblasts. Because calcium forms an Alizarin Red S-calcium complex in a chelation process, with the end product producing birefringent, Alizarin red staining can be used to assess a cellular function like bone formation. Also cellular function can be analyzed by proteomic or genomic assays by quantifying bio-reaction products or cellular proliferation, as known in the art.

### E. Illustrative Products

The nanoparticle compositions provided herein may be used in a variety of products, including but not limited to compositions, nutraceuticals, kits, gels, creams, reagents, implants, scaffolds, injectables, inhalants, surface coatings for implantable medical devices such as catheters, tubes, dental implants, orthopedic implants, orthopedic devices to include plates, screws, pins, rods, and in cardiovascular applications such as defibrillators and stents, tissue engineering constructs, cell culture dishes, and related tools.

Specific examples are presented below of methods for enhancing a cellular function. They are exemplary and not limiting.

### Primary Culture Preparation

Bone cells were plated in 100 mm culture dishes in a density of 10⁶/dish and were supplemented by α—Minimum Essential Medium with 10% FBS and 1% PS and incubated in 37 C, 5% CO₂ humidified incubator. When the cells were at confluence, the cells were trypsinized by 1× trypsin.

Osteoblast cells were plated at a desired density in 24 well plates; 10⁵/well and incubated with 1 ml α—Minimum Essential Medium with 10% FBS and 1% PS with or without nanoparticles (10 μg/ ml) of Ag-NPs, Hydroxyapatite nanoparticles, TiO₂ nanoparticles and CNTs; incubated in 37 C in a 5% CO2 humidified incubator for 6 days until cells were confluent and the medium was necessarily changed every 48-72 hrs by aspirating half the volume and adding 0.5 ml of fresh medium for each well.

### Osteogenesis Induction

The medium was aspirated completely and replaced with 1 ml of Osteogenesis Induction Medium #1, containing approximately 99% cell culture medium, 0.02 mM/ml Ascorbic Acid 2—Phosphate solution, and 1 mM/ml Glycerol 2—Phosphate solution. This medium change corresponded to differentiation day 0 and was changed with 1 ml fresh Osteogenesis Induction Medium #1 every 2-3 days.

On differentiation day 9, the medium was replaced with 1 ml fresh Osteogenesis Induction Medium #2 by adding 5 nM/ml Melatonin solution to the Osteogenesis Induction Medium #1. The medium was replaced with fresh Osteogenesis Induction medium #2 every 2-3 days.

Osteogenesis Quantification Assay

After 24 days, the cells were fixed with 10% formaldehyde for 10 minutes; washed 3 times, 5-10 minutes each with 1× Phosphate Buffer Saline, and stained with Alizarin Red Stain solution by adding 400 μl for each well and incubated for 30 minutes. The stain was drained and the cells were washed 3 times with 1×]PBS 5-10 minutes each.

For osteogenesis quantification, 400 μl 10% acetic acid was added to each well and incubated for 30 minutes with shaking to loosen the attachment of the monolayer with the aid of a cell scraper. The cells and acetic acids were transferred to 1.5 ml micro-centrifuge labeled tubes and vortexed vigorously for 30 seconds. The samples were heated at 85 C for 10 minutes and transferred directly to ice for 5 minutes. The samples were then centrifuged at 20,000 g for 15 minutes.

Alizarin Red standard solution was made by diluting 10×_9 ARS dilution buffer 1:10 in distilled water to obtain 1× ARS dilution buffer and then 40 mM Alizarin Red Stain solution was diluted 1:20 in 1× ARS dilution Buffer to yield 2 mM working stock.

After centrifuging the samples, 400 μl of the supernatant was removed and transferred to new 1.5 ml microcentrifuge tubes and 150 ∞l of Ammonium hydroxide solution was added to each tube to neutralize the pH and ensure it fell within the range of 4.1-4.5.

400 μl of the standard sample was placed in a spectrophotometer cuvette and read at OD₄₀₅ and the Alizarin Red stain concentration in each sample was plotted vs. OD₄₀₅.

The Alizarin Red concentration in each sample was calculated according to the OD of the standard solution and the machine was calibrated by blank solution using 400 μl of 1× ARS dilution buffer.

The effect of nanomaterials on the concentration of Alizarin Red stain: the osteoblast cells were incubated in the presence and absence of (10 μg/m1) of Ag-NPs, HAP nanoparticles, TiO₂ nanoparticles and CNTs. The experiments were completed on day 24. The results were derived from three experiments, with 6 cultures for each variable in each experiment (n=6). Alizarin Red concentration was determined by comparing the samples OD₄₀₅ with a standard sample of 2 mM of ARS diluted with 1× ARS dilution buffer.

TABLE 1
Effect of different nanomaterials on the Alizarin Red concentration

| | | Alizarin Red Concentration |
|---|---|---|
| Sample | OD405 (nm) | (mM) |
| Control | 1.4983 ± 0.1310 | 0.7684 |
| Ag—NPs | 4.1283 ± 0.0734 | 2.1171 |
| HAP | 2.9950 ± 0.1513 | 1.5353 |
| TiO₂ | 2.2967 ± 0.2142 | 1.1778 |
| CNTs | 1.7050 ± 0.1191 | 0.8744 |

The mean of Alizarin Red concentration for each variable was determined from the OD₄₀₅ in 6 cultures and three experiments. The measurements were taken from day 24 of the experiment after the osteoblasts were incubated at the desired concentration of different nanomaterials.

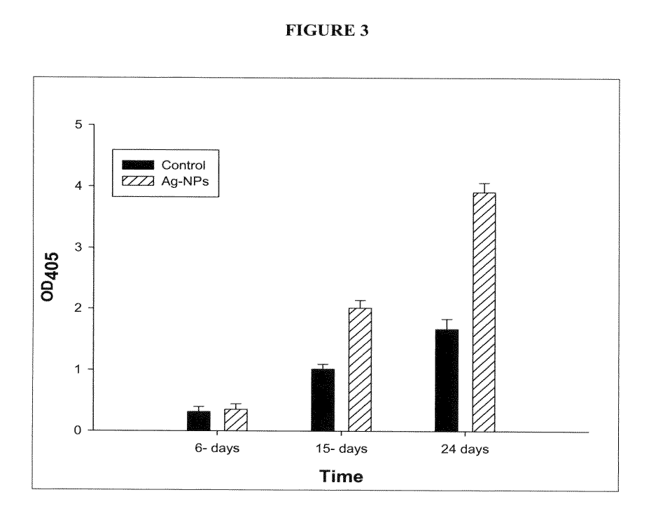

Effect of silver nanoparticles on the concentration of Alizarin Red stain as a function of time: 10⁵ cells were plated per well with and without silver nanoparticles (10 μg/ml) and incubated for 6, 15, and 24 days. The results were derived from 3 experiments, with 6 cultures for each variable in each experiment. Bars in FIG. 3 represent the OD₄₀₅ which is correlated with the Alizarin Red concentration in each well.

TABLE 2
Effect of Ag—NPs (10 μg/ml) on Alizarin Red conc.

| Time (Days) | Control | Ag—NPs |
|---|---|---|
| 6 | 0.3133 ± 0.0827 | 0.3517 ± 0.1754 |
| 15 | 1.0133 ± 0.0803 | 2.0100 ± 0.2473 |
| 24 | 1.6683 ± 0.1641 | 3.9067 ± 0.2984 |

Table 2 shows the results are the mean±SD of the OD₄₀₅ of six wells for each variable derived from 3 experiments (six wells per experiment).

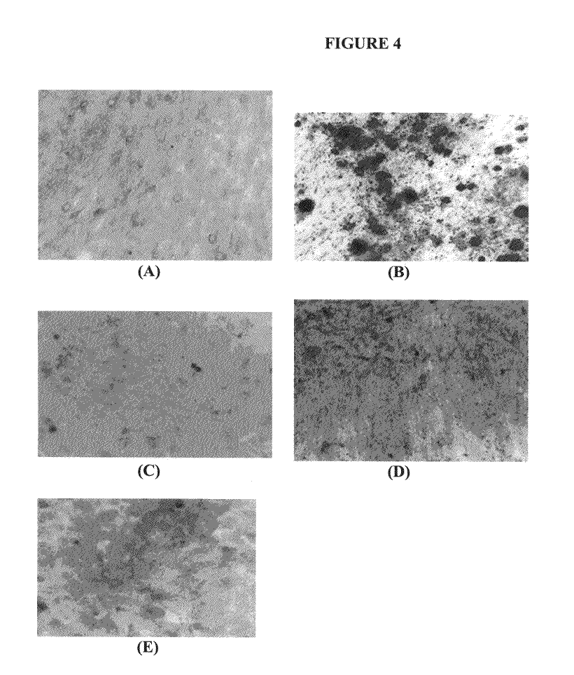

Images in FIG. 4 of the mineralization nests (colored in red under various experimental conditions when the cells were exposed to identical quantities of various nanomaterials): the mineralized nodules formation of osteoblasts in the presence of nanomaterials stained by alizarin red S. (A) cells without nanomaterials as a control; (B) cells with Ag-NPs; (C) cells with HAP nanoparticles, (D) cells with TiO₂ nanoparticles; (E) cells with SW-CNTs. Original magnification 40×, bar=50 μm. The more red the image is, the more mineral has formed after exposing the cells to the various nanomaterials.

Information regarding the in vivo process of mineralization, as well as the evaluation of osteogenic cellular response to implant materials can be provided by studying an osteoblast cell-culture system which is able to form an extracellular matrix capable of mineralization.

Various methods, such as controlling cell density, using enriched media, and addition of various nanomaterials, have been used to alter culture conditions to modulate or enhance mineralization.

Bone is constantly reshaped, the osteoblasts building bone and the osteoclasts resorbing bone. An osteoblast is a mononucleate cell that is responsible for bone formation and mineralization of the osteoid matrix. Primary osteoblast cells have been shown to be a model system for revealing biological effects of nanomaterials because their susceptibility to nanomaterials is similar to that in vivo.

The use of nanomaterials in bone-culture systems was introduced based on the observation that nanoparticles passed through cell membranes and altered cell functions such as mineralization, protein synthesis, gene expression, etc. The level of bone deposition or mineralization of newly formed bone is generally considered to be closely related to the activity of alkaline phosphatase.

The objective of the present work was to investigate the biological effects of Ag-NPs, TiO₂ nanoparticles HAP nanoparticles, and SW-CNTs, in vitro by quantification of mineralized nodule formation in the osteoblast culture system in vitro.

The order of the enhancing effect is Ag-NPs>HAP nanoparticles>TiO₂ nanoparticles>SW-CNTs>control. CNTs inhibited the formation of the mineralized nodules greatly.

What is claimed is:

1. A method for regulating a cellular function, comprising
(a) combining a material and a nanoparticle composition to form a material system, wherein said combining is by coating or binding; and
(b) delivering said material system to a cell type or tissue.

2. The method of claim 1, wherein said material is for a biomedical application.

3. The method of claim 1, wherein said material is at a nano, micro, or macro scale.

4. The method of claim 1, wherein said nanoparticle composition comprises gold, metal, metal oxide, polymer, titanium dioxide, silver, carbon nanotubes, hydroxyapatite, quantum dots, crystals, salts, ceramic materials, magnetic materials, or any combination thereof.

5. The method of claim 1, wherein said nanoparticle composition comprises a plurality of materials.

6. The method of claim 5, wherein said plurality comprises
(a) a core made of one material; and
(b) at least one layer surrounding said core, wherein said layer comprises a material that is not the same as said core.

7. The method of claim 1, wherein said nanoparticle composition comprises material having a size from about 0.5 nm to about 50 mm

8. The method of claim 6, wherein said material has a size from about 50 microns to 50 mm.

9. The method of claim 1, wherein said material is coated with at least one layer of coating that delivers a drug or bioactive agent, wherein said drug or bioactive agent prevents oxidation, prevents or treats infection, increases bio-compatibility, increases or decreases a cell function, promotes cell adhesion and proliferation, reduces toxicity, or promotes binding with a biological or non-biological system.

10. The method of claim 1, wherein said delivering is by injection, epidermal translation, inhalation, surgical delivery, topical application, oral administration or any other method that targets a desired cell type or tissue.

11. The method of claim 1, wherein said cellular function is at least one of bone formation, protein synthesis, gene expression, cell proliferation, mitosis, DNA transcription, hormone production, enzyme production, cell death, gene delivery, or drug delivery.

12. The method of claim 1, wherein said material is a natural or synthetic polymer, metal, metal oxide, metal nitride, borate, ceramic, allograft hard tissue, allograft soft tissue, xenograft hard tissue, xenograft soft tissue, carbon nanostructure, glasses, or natural, or biocompatible material.

13. The method of claim 1, wherein said material is coated with an oxide,nitride, borate, polymer, ceramic, zirconia, metal, metal oxide, carbon, or graphitic material.

14. The method of claim 1, wherein said material system is capable of preventing or treating an infection.

15. The method of claim 14, wherein said infection is a bacterial, fungal, prion, parasite, or viral infection.

16. The method of claim 12, wherein said carbon nanostructure is a single walled carbon nanotube, double walled carbon nanotube, multi walled carbon nanotube, nanofiber, fullerene, or grapheme.

17. The method of claim 1, wherein said tissue is bone tissue.

18. The method of claim 1, wherein said tissue is soft tissue.

19. A method for regulating a cellular function, comprising delivering nanoparticles in a solution, aerosol, gel, cream, paste to a cell type or tissue.

20. The method of claim 19, wherein said delivery is by injection, inhalation, oral administration, or topical application.

21. A device for regulating at least one cellular function, wherein said device comprises a material system.

22. The device of claim 21, wherein said device is an implant, graft, needle, catheter, dental implant, gel, cream, injectible, orthopedic implant, prosthetic, cardiovascular stent, defibrillator/pacemaker, medical tube, tissue engineering matrix or scaffold.

23. A device comprising nanoparticles, wherein said nanoparticles are layered throughout and/or on the surface of said device.

24. The device of claim 23, wherein said device is biodegradable and/or biocompatible.

25. The device of claim 23, wherein said nanoparticles are released from said device as each layer degrades.

26. The device of claim 22, wherein said scaffold is a tissue-forming scaffold.

27. A composition for promoting tissue growth, wherein said composition comprises nanoparticles.

28. The composition of claim 27, wherein said nanoparticles are layered in a surface coating covering.

29. The composition of claim 27, wherein said nanoparticles comprise gold, silver, metals, metal oxides, oxides, polymers, ceramics, carbon nanotubes, or hydroxyapatite.

30. A coating for an implant; wherein said coating comprises nanoparticles.

31. The coating of claim 30, wherein said implant is polymeric, metal, metal alloy, shape memory metal, metal oxide, oxide, ceramic, zirconia, or hydroxyapatite.

32. A method for regulating a cellular function, comprising
(a) linking at least one nanoparticle to an agent to create a stimulating agent; and
(b) delivering said stimulating agent to a cell type or tissue.

33. The method of claim 32, wherein said agent comprises a protein, growth factor, antibody, amino acids, polymers, drug(s), hormone, nucleic acid, peptide, or enzyme.

34. The method of claim 32, wherein said cellular function is bone growth and said stimulating agent is an osteoblast stimulating agent.

35. The method of claim 32, wherein said linking is covalent, polar covalent, ionic, sulfide, hydrogen bond, or any other linkage suitable for in vivo.

36. The method of claim 33, wherein said growth factor is Bone Morphogenic Proteins (BMPs), Brain-Derived Neutrophic Factor (BDNF), Ciliary Neutrophic Factor (CNTF), Epidermal Growth Factor (EGF), Erythropoietin (EPO), Fibroblast Growth Factor (FGF), Granulocyte-Colony Stimulating Factor (G-CSF), Granulocyte-Macrophage Colony Stimulating Factor (GM-CSF), Growth Differentiation Factor-9 (GDF9), Hepatocyte Growth Factor (HGF), Insulin-like Growth Factor (IGF), Interleukin (IL), Leukemia Inhibitory Factor (LIF), Myostatin (GDF-8), Nerve Growth Factor (NGF), Neutrophic Factors (NT), Platelet-derived Growth Factor (PDGF), Thrombopoietin (TPO), Transforming Growth Factor alpha(TGF-α), Transforming Growth Factor beta (TGF-β), or Vascular Endothelial Growth Factor (VEGF).

37. The method of claim 32, wherein said delivering is by injection, surgical placement, oral administration, inhalation, or transdermal application.

38. The method of claim 32, further comprising (c) exposing said cell or tissue comprising said stimulating agent to radiation for faster penetration.

39. The method of claim 38, wherein said radiation is laser radiation or electromagnetic radiation.

40. The method of claim 32, wherein said stimulating agent is placed in a solution, gel, paste, suspension, or aerosol, for delivery to said cell type or tissue.
